(19) United States
(12) Patent Application Publication
(10) Pub. No.: US 2009/0209423 A1
(43) Pub. Date: Aug. 20, 2009

(54) CORN PROTEIN CONCENTRATES

(76) Inventors: Johannis Slabbekoorn, Oostburg (NL); Johan De Meester, Antwerpen (BE); Cornelius Schot, Sint-Catharinaplein (NL); Annemiek Van Cauteren, Bavel (NL); Rita Delrue, Bergen op Zoom (NL); Eugene J. Fox, Dayton, OH (US); Donald L. Shandera, JR., Ashland, NE (US); Charles Anderson, Hereford, TX (US)

Correspondence Address: FISH & RICHARDSON P.C., PO BOX 1022, MINNEAPOLIS, MN, 55440-1022, US

(21) Appl. No.: 11/997,833

(22) PCT Filed: Aug. 2, 2006
(86) PCT No.: PCT/US06/30114

§ 371 Date: Dec. 24, 2008

Related U.S. Application Data
(60) Provisional application No. 60/705,085, filed on Aug. 3, 2005.

(51) Int. Cl. A01N 25/00; C07K 2/00; A01P 13/00; C05F 11/00

(52) U.S. Cl. 504/101; 530/300; 504/358; 71/23

(57) ABSTRACT

The invention provides for corn protein concentrates (CPC). The CPC described here can be used for herbicidal, fertilizer, and nutrient media benefits.

### TECHNICAL FIELD

This invention relates to protein concentrates, and more particularly to corn protein concentrates.

### BACKGROUND

Corn wet milling is used to separate corn kernels into products such as starch, protein, fiber and oil. Corn wet milling is a two stage process: a steeping process to soften the corn kernel and to facilitate the next step; and a wet milling process resulting in purified starch and different co-products such as oil, fiber, and protein. The purified protein stream is corn gluten meal (CGM) and is typically sold on a 60% protein as-is basis. The allowable composition of corn gluten meal utilized for animal feed is defined by American Feed Control Officials, Inc. (AAFCO) Feed Ingredient Definition 48.14. Under this definition, CGM may contain fermented corn extractives and/or germ meal. CGM typically has a pH near 4 similar to the wet milling process. It contains significant concentrations of organic acids, primarily lactic acid, and has an odor representative of the milling process. Corn gluten meal composition can vary substantially between manufacturers and plants.

Pest control and nutrient supplementation of horticultural and agricultural applications are typically accomplished with chemicals and synthetic substances. Few natural herbicide alternatives exist for controlling weed growth in lawns, gardens, and fields. Lawns, golf courses, other turf grasses, and gardens are typically intensively managed to control pests and visual quality. Corn gluten meal can be used for retarding growth of weeds and for its nitrogen content. Organic and natural alternatives for herbicide and fertilizer applications are needed to retard unintentional consequences on non-target organisms and long-term ecological effects of synthetic chemicals.

### SUMMARY

The invention provides for corn protein concentrates (CPC). The CPC described herein can be used for purposes such as for fertilizers, herbicides, and mushroom media.

In one aspect, the invention provides for a herbicide product that includes a corn protein concentrate incorporated on or in the herbicide. In another aspect, the invention provides for a fertilizer product that includes a corn protein concentrate incorporated on or in the fertilizer. A corn protein concentrate according to the invention is prepared by a process that includes contacting one or more protein containing materials with one or more wet-mill streams and one or more carbohydrases, which produces at least one protein concentrate and at least one aqueous stream containing water-soluble carbohydrates, and separating the protein concentrate from the aqueous stream containing water-soluble carbohydrates. The process of making a corn protein concentrate as described herein further can include defatting the protein-containing material; decoloring, bleaching, and/or reducing the color-bodies present in the protein-containing material; contacting the corn protein concentrate with a deodorizing compound (e.g., a cyclodextrin); and/or contacting the one or more protein-containing materials with one or more phytases.

In a process of making a corn protein concentrate as described herein, the one or more wet-mill streams can include steep liquor, light steep water, heavy steep liquor, or mixtures thereof; the wet-mill stream can be derived from a gluten concentrating or mill thickening wet-mill stream such that the majority fraction of the mill stream is of a nitrogenous or protein content; the protein-containing material can be light gluten fraction, heavy gluten fraction, corn gluten concentrate, corn gluten meal, gluten cake, and mixtures thereof; and the carbohydrase can be alpha amylase, dextrinase, pullulanase, glucoamylase, hemicellulase, cellulase, and mixtures thereof.

In yet another aspect, the invention provides a herbicide and/or fertilizer product that includes a corn protein concentrate that is prepared by a process that includes contacting one or more protein containing materials with one or more wet-mill streams and one or more carbohydrases, which produces at least one protein concentrate and at least one aqueous stream containing water-soluble carbohydrates, and separating the protein concentrate from the aqueous stream containing water-soluble carbohydrates.

In another aspect, the invention provides a corn protein concentrate that has at least about 80% protein on a dry weight basis. In one embodiment, the corn protein concentrate substantially lacks one or more exogenous polypeptides having saccharification enzyme activity. Such exogenous polypeptides are generally derived from microorganisms (e.g., fungi and bacteria) and include glucoamylases, pullulanases, or mixtures thereof.

In yet another aspect, the invention provides for a corn protein concentrate having at least about 80% protein on a dry weight basis and a carbohydrate profile wherein at least 10% of the water extractable carbohydrates DP 5-13 (total 5-13) as percent of DP 1-13 (total area 1-13).

In another aspect, the invention provides for a corn protein concentrate wherein, when the corn protein concentrate is compared to corn gluten meal, the corn protein concentrate a) has a higher and more consistent pH; b) has a lower wet milling odor; c) exhibits less bacterial counts; d) releases less protein into a 0.5 N NaOH solution; e) releases less protein into an SDS solution; and/or f) has a lower water activity.

In other aspects, a corn protein concentrate according to the present invention, when compared to corn gluten meal: a) has a pH that consistently stays above about 5; b) has a lower wet milling odor; c) exhibits less bacterial counts; and/or d) has a lower ash content on a per protein basis.

In still other aspects, the invention provides a corn protein concentrate that has a lower water activity than corn gluten meal at a moisture content of less than 10%; and a corn protein concentrate including at least about 80% protein on a dry weight basis, less than about 5% of granular starch and about 1% to about 10% liquefied starch carbohydrates and sugars. Generally, at least 10% of the total water extractable carbohydrates (DP 1-13) in a corn protein concentrate as described herein come from the liquefied starch carbohydrates (DP 5-13).

In yet another aspect, the invention provides a corn protein concentrate, wherein, when the corn protein concentrate is compared to corn gluten meal following extrusion, the corn protein concentrate: a) exhibits more controllable expansion; b) exhibits greater expansion; c) exhibits more uniform cell structure; d) creates a more homogenous product; e) produces a kibble with a smoother surface; and/or f) exhibits greater oil binding capacity.

In another aspect, the invention provides for a corn protein concentrate that has been treated with acid and/or proteases to produce partial or complete hydrolysis of the protein. The hydrolyzed protein may optionally be heated in the presence of a sugar or other compound.

In another aspect, the invention provides for a protein concentrate that is contacted with and optionally dried in the presence of another compound to reduce odor. In some embodiments, the corn protein concentrate is treated with cyclodextrins to produce a corn protein concentrated with very little wet milling odors and a bland smell.

In another aspect, the invention provides for a protein concentrate to be used as an ingredient in mushroom cultivation. The corn protein concentrate provides a nutrient source to the growth media.

The corn protein concentrate of the present invention provides several benefits in comparison to corn gluten meal. For example, the corn protein concentrate provides a higher nitrogen concentration for fertilizer applications without the starch component resulting in lower application rates with lower visual presence of the corn protein concentrate in the application than corn gluten meal. Additionally, the corn protein concentrate of the present invention may lower microbial growth preventing acidification of soils due to the associated degradation of the starch and higher and more immediate plant availability of the nitrogen from the corn protein concentrate than from corn gluten meal due to less microbial growth using the products nitrogen for associated growth. The higher pH of corn protein concentrate in comparison to corn gluten meal will also reduce acidification effects on soil pH. Due to the lower water uptake, corn protein concentrate will also provide slower release than corn gluten meal.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DETAILED DESCRIPTION

The present invention provides for a corn protein concentrate that can be used for industrial and domestic purposes such as for fertilizers and herbicides.

### Corn Protein Concentrate (CPC)

A CPC described herein generally has at least 80% protein (on a dry weight basis) (e.g., 85%, 90%, 95%, 99%, or 100% on a dry weight basis). The CPC described herein is composed primarily of prolamines and glutelins based on the Osbourn Classification System of classifying proteins, which is based on the solubility or polypeptides in a solvent. A typical proximate analysis of a CPC described herein compared to corn gluten meal (CGM) is shown below.

| | CPC | CGM |
|---|---|---|
| Component | (as is) | (as is) |
| Protein | 75% | 60% |
| Fat (Ether Extractable) | 2.5% | 2% |
| Crude Fiber | 3% | 2.5% |
| Starch | <1% | 15% |
| Moisture | 10% | 10% |
| Xanthophyll (mg/lb) | 121 | 107 |
| Density (lb/cu ft) | 38-41 | 36-43 |

A typical amino acid analysis of CGM is shown below. The amino acid composition of a CPC described herein are not expected to differ significantly from that of CGM.

| Amino Acid | % of Total | % of Protein |
|---|---|---|
| Alanine | 5.94 | 7.92 |
| Arginine | 2.42 | 3.23 |
| Aspartic Acid | 4.40 | 5.87 |
| Cystine | 1.13 | 1.50 |
| Glutamic Acid | 15.5 | 20.7 |
| Glycine | 1.69 | 2.25 |
| Histidine | 1.76 | 2.35 |
| Isoleucine | 3.15 | 4.20 |
| Leucine | 12.9 | 17.3 |
| Lysine | 1.08 | 1.45 |
| Methionine | 1.59 | 2.12 |
| Phenylalanine | 4.65 | 6.20 |
| Proline | 6.95 | 9.27 |
| Serine | 4.07 | 5.42 |
| Threonine | 2.21 | 2.95 |
| Tryptophan | 0.30 | 0.40 |
| Tyrosine | 4.22 | 5.62 |
| Valine | 3.44 | 4.59 |

As used herein, CGM refers to the dried residue from corn after the removal of the larger part of the starch and germ and the separation of the bran by the process employed in the wet milling manufacture of corn starch or syrup, or by enzymatic treatment of the endosperm. CGM may contain fermented corn extractives and/or corn germ meal. The protein in CGM has low solubility in water.

Dry solids can be determined by drying of the material at 103° C. using a method adapted from Dutch standard method NEN 3332 and according to the American Association of Cereal Chemists (AACC) Official Method 44-15A or by using Official Methods of the AOAC International (AOAC), sec. 935.29.

Protein content of CPC in solution can be determined using, for example, a Bradford Protein Assay (Bradford, 1975, Anal. Biochem., 7:248). Total and soluble protein content can be determined according to AACC Method 46-30 or AOAC 990.03. Starch content can be determined using a method derived from suitable official analytical methods such as Corn Refiners Association's (CRA) G-28. Total starch and liquefaction-produced carbohydrates can be determined by the AOAC Official Methods of Analysis 996.11. Liquefaction products of starch hydrolysis are not intact starch and should be considered as liquefaction products composed of soluble starch, higher sugars, and sugars. These can be separated from the analysis by methods such as washing with water and/or washing with ethanol. The difference between starch compositional results of CRA G-28 and AOAC 996.11 (e.g., the difference between measured total starch carbohydrates and starch content) results in the amount of soluble starch, higher sugars, and sugars, which should be considered starch liquefaction products instead of the sugars native to the mill streams.

The sugar content of the mill streams and the collected filtrate can be determined using a procedure derived from AACC Method 80-05 (e.g., using a HPLC system (e.g., Aminex BPX-87H ion exclusion column (Bio-Rad, Hercules, Calif.)) eluded with 0.01 N sulfuric acid mobile phase and having a refractive index detector). The sugar content generally is the sum of the amount of glucose, fructose, maltose and maltotriose sugars standardized against the column.

Sugar DP profile and quantitation in mill streams, liquefact, and extracted solubles of CPC can be performed using a procedure derived from AACC Method 80-05. The water extractables can be analyzed by, for example, precipitating proteins with sulfosalacylic acid, ion exchanging with anion and cation resin, filtering each liquid fraction through a filter (e.g., 0.45 micron Whatman syringe filter), and injecting the liquid into an HPLC system having a silver ion exchange column with water as the mobile phase and having a refractive index detector.

Analysis of the obtained information can be made as the sum of the eluded peaks less than the degree of glucose polymerization (DP) of 14 standardized against the column. The percentage of DP 1-4 sugars (calculated as the sum of the area under the curve of DP 1-4 sugars divided by the sum of the area under the curve of DP 1-14 sugars) is compared to the percentage of DP 5-13 sugars (calculated as the sum of the area under the curve of DP 5-13 sugars divided by the sum of the area under the curve of DP 1-14 sugars). Generally, CGM has predominantly DP 1-2 sugars with only trace amounts, if any, of DP 5-13. On the other hand, the CPC disclosed herein can contain about the same amount or a higher amount of DP 5-13 sugars than DP 1-4 sugars.

Total or crude lipid content can be determined using a protocol derived from AACC Methods 30-24, 30-20, 30-25, CRA G-11, or by AOAC 920.39 or 954.02. Methods using ether extraction, hexane extraction incorporating ball milling in a Spex mill, or acid-hydrolysis often result in different lipid values, with ether extraction generally resulting in the lowest lipid values and acid-hydrolysis generally resulting in the highest lipid values.

The water- or alcohol-absorption can be determined using absorption indexes and the water- or alcohol-solubility can be assessed using Osbourn's classification of protein extraction and solubilization scheme.

Organic acid content can be determined by HPLC using UV or RI detection, such as using a HPLC system (e.g., Aminex BPX-87H ion exclusion column (Bio-Rad, Hercules, Calif.) eluded with 0.01 N sulfuric acid mobile phase and a refractive index detector.

Ash can be determined using a procedure derived from AACC Method 08-01 by wet-ashing of a sample at 560° C. or by AOAC 942.05.

Crude fiber can be determined by AOAC 962.09.

Phytate can be determined in a sample by extraction of phytic acid, which can be purified using different techniques and analyzed quantitatively by HPLC using conductivity.

Water activity (a_{w}) is the relative availability of water in a substance and is defined as the vapor pressure of water divided by that of pure water at the same temperature. For example, pure distilled water has a water activity of 1.0. As the temperature increases, a_{w} typically decreases, with the exception of some salt and sugar solutions. Water tends to migrate from high a_{w} substances to low a_{w} substances. In addition, higher a_{w} substances tend to support more microorganism growth. For example, bacteria usually require an a_{w} of at least 0.91 and fungi at least 0.7.

a_{w}≡p/p₀

where p is the vapor pressure of water in the substance, and p₀ is the vapor pressure of pure water at the same temperature.

### Methods of Making a Corn Protein Concentrate

The CPC described herein can be made by the process described in PCT Application No. PCT/US2005/003282, which is incorporated herein by reference in its entirety. Briefly, a CPC described herein is prepared by a process that includes contacting one or more corn protein-containing materials with one or more wet-mill streams and one or more carbohydrases.

The term “corn protein-containing material” refers to streams generated from the wet-milling process wherein greater than 2% of the solids are gluten and less than one quarter of the original kernel fiber and germ. The term “corn gluten” as used herein refers to water insoluble proteins derived from endosperm. Corn protein-containing material includes streams such as heavy gluten, gluten cake, starch wash overflow, and primary feed. One or more of these corn-protein-containing materials can be used in the process.

A wet-mill stream is a flowable stream formed by the wet-milling process. Exemplary wet-mill streams include corn steep liquor (CSL), which can be either heavy (evaporated CSL) or light (LSW), primary feed, any centrifuge or hydrocyclone overflow, a washing or dewatering filtrate, or mixtures thereof. Examples of centrifuge overflows include mill stream thickener overflow, primary overflow, clarifier overflow, starch wash overflow, or mixtures thereof. Examples of hydrocyclone overflows include starch wash overflow and millstream thickener. Examples of washing and dewatering streams include gluten filtrate and fiberwash filtrate. These streams are characterized in that they have at least trace amounts of protein and carbohydrates from corn.

The carbohydrases used can be any enzyme that can facilitate the degradation (such as by saccharification and/or liquefaction) of a complex carbohydrate to a water-soluble carbohydrate. For example, enzymes such as alpha-amylases, glucoamylases, dextrinases, pullulanases, hemicellulases, and cellulases or mixtures can be used. Alpha-amylase can be used to liquefy starch up to about a 40 dextrose equivalent (DE) sweetness measure. Mixtures of glucoamylase and pullulanase can be further used in a saccharification step after liquefaction to further degrade the starch polymers up to about 95-97DE, which contain greater than 90% of the total sugars (DP 1-14) with a composition of at least 90% sugars of DP 1-4.

In some embodiments, the methods involve liquefaction without saccharification. In these embodiments, the enzymes used will be those commonly used to hydrolyze starch molecules such as alpha-amylases. In some embodiments, the methods involve contacting the material with hemicelluloses and celluloses in combination with liquefaction and, optionally, saccharification. Malted grain and parts thereof may also be used as a source of enzyme.

In some embodiments, the protein content of the protein concentrate can be altered by using additional enzymes. For example, phytases and/or pectinases can be used to digest the phytate and/or the pectin, respectively, which will allow them to be separated from the protein concentrate. Use of phytases and pectinases may also result in a protein concentrate that is more digestible than a concentrate that has not been treated.

In some applications, elongated proteins are more desirable. Enzymes that join protein fragments such as polyphenoloxidases and/or transglutaminases can be used. These enzymes can be introduced simultaneously with the carbohydrases or they can be added in a separate step.

The corn protein-containing material(s), the wet mill-stream(s), and the carbohydrases can be placed in contact with each other using any method known in the art, such as by slurring, mixing, or blending. In some embodiments, methods can include a filtration step to remove unwanted or undesirable components.

The composition containing the carbohydrases, wet-mill stream(s), and corn protein-containing material(s) is incubated at a time and temperature sufficient to at least degrade the starch and/or other complex carbohydrates present in the corn protein-containing material and/or the wet-mill stream to the point where, upon separation of the aqueous stream containing water-soluble carbohydrates from the resulting corn protein concentrate, the aqueous stream has a higher concentration of water-soluble carbohydrates then the wet-mill stream had prior to contacting the carbohydrases.

Exemplary temperatures that can be used to incubate the mixture containing the carbohydrases, wet-mill stream(s), and corn protein-containing material(s) include from about 30 to about 250° F. (15-120° C.), and exemplary incubation times include from about ½ hours to about 40 hrs. The incubation temperature and time depend on the starting materials, enzymes, and the amount of enzymes used.

Separating the corn protein concentrate from the aqueous stream can be accomplished by any method known in the art. For example, filtration, centrifugation, coagulation, and combinations thereof can be used. It is also possible to increase the concentration of water-soluble carbohydrates in the aqueous stream by recycling or reusing the aqueous stream as one of the wet-mill streams used in the process.

The concentration of protein in the resulting protein concentrate can additionally be increased by rinsing the resulting concentrate with water and/or a wet-mill stream. The rinsing washes away residual carbohydrates and increases the protein concentration on a dry basis. Using this technique, the protein concentration can be increased by at least 2%, 5%, 7%, 10%, or 20% on a dry basis.

Yet another way of increasing the concentration of protein in the protein concentrate is to remove fats from the concentrate (i.e., defatting). Defatting can be accomplished using any method known in the art, for instance by using one or more solvents and/or enzymes to degrade the fats. Examples of solvents that can be used include hexane, isohexane, alcohols, and mixtures thereof. Examples of enzymes that can be used include lipases and the like. The fats can subsequently be separated from the protein concentrate using any method known in the art, for example filtration, floatation, and/or centrifugation.

Additionally, a protein concentrate can be decolorized by bleaching using either chemical and/or enzymatic methods. Enzymes that can be used to facilitate bleaching include those having lipoxygenase (LOX) activity or peroxidase activity. Chemicals that can be used alone or in combination with enzymes to facilitate bleaching include ozone, persulfate, and peroxides.

The filtration of the protein concentrate can be accomplished while the stream containing the protein is at temperatures of, for example, greater than 45° C., 50° C., 55° C., 60° C., 65° C., 80° C., or 100° C. This provides the advantage of being able to control microbial growth and mycotoxin concentration during the filtration process. The ability to use increased temperatures also allows enzyme activity to be modulated.

A CPC as described herein can be treated with an acid (e.g., in the presence of heat and/or pressure) and/or treated with one or more proteases. The one or more proteases can possess general hydrolyzing activity on peptide bonds or the one or more proteases may possess a more specific activity such as, for example, enhancing a processing functionality or generating a flavor. Hydrolyzed proteins optionally can be heated in the presence of a sugar (e.g., a reducing sugar such as glucose, fructose, corn syrup, or other compound) to produce a desirable smell and flavor. For example, a meaty flavor can be generated by heating the amino acid, valine, in the presence of a reducing sugar. Alternatively, proteins can be deaminated by such treatments to alter functionality such as water solubility. A CPC can be deodorized by using deodorizing compound. A deodorizing compound can be added to CPC in a dry state or in a liquid or slurried state. Deodorizing compounds include, without limitation, cyclodextrins and alcohols. Examples of cyclodextrins include, without limitation, alpha-cyclodextrins, beta-cyclodextrins, and/or gamma-cyclodextrins. Cyclodextrins can be modified by substituting functional groups, such as hydroxypropylated, methlated, ethylated, or acethylated with various levels of substitution to yield different activities that result in distinct odors and solubility. The deodorizing compound can be introduced at any point during the process of making CPC. The deodorizing compound can be added to the finished CPC product or can be applied to the CPC packaging by mixing, blending, spraying, coating, or other methods obvious to those skilled in the arts. Although the amount of a deodorizing compound that is used in a CPC can vary, generally about 0.05% to 5% (wt/wt CPC on a dry basis) (e.g., about 0.25% to 2.5% (wt/wt CPC on a dry basis)) of a deodorizing compound will provide sufficient result. Similar results may be obtained by using deodorizing compounds on CGM with higher application rates.

In some embodiments, CPC may optionally be treated with acid and/or proteases to produce a partial or complete hydrolysis of the protein. The hydrolyzed protein may optionally be heated in the presence another compound. Hydrolyzed CPC may be applied in a liquid form or may be dried or dried onto/with another compound and applied in its dry form. Hydrolyzed CPC may be slurried and dried in the presence of deodorizing compounds such as cyclodextrins to alter odor.

### Fertilizers and Herbicides

The CPC described herein can be used in horticultural, agricultural, and industrial applications, including as fertilizers and/or herbicides for vegetation. Vegetation includes, without limitation, grasses, lawns, gardens, fungus, crops (corn, wheat, beans), shrubs, or trees.

The CPC of the present invention provides several benefits in comparison to CGM. For example, the CPC provides a higher nitrogen concentration for fertilizer applications without the majority of the starch component resulting in typically 15-25% lower application rates with lower visual presence of the CPC in the application than CGM. Additionally, the CPC of the present invention may help prevent acidification of soils due to: 1) a higher, more neutral pH of the CPC with lower organic acids, and 2) lower microbial growth to the associated degradation of the starch. In another aspect, more immediate plant availability of the nitrogen from the CPC than from CGM is expected due to less microbial growth using the products nitrogen for associated growth resulting from the starch carbohydrates present in the soil. Due to the lower water uptake, CPC will also provide slower release than CGM. CPC can also be applied at high application rates without burning vegetation as compared to inorganic fertilizers.

Fertilizer benefits may be accomplished by applying to a soil or vegetation an effective amount of CPC. The amount of CPC that can be applied can vary over a wide range, but typical application rates are about 1 lb/1000 sq. ft. to about 100 lb/sq. ft., and more preferably from about 8 lbs/1000 sq. ft. to about 25 lbs/1000 sq. ft.

Herbicidal benefits may be accomplished by applying to a soil or vegetation an effective amount of CPC. The amount of CPC that can be applied can vary over a wide range, but typical application rates are about 1 lb/1000 sq. ft. to about 100 lb/sq. ft., and more preferably from about 8 lbs/1000 sq. ft. to about 25 lbs/1000 sq. ft.

CPC can be applied to its respective application by employing an conventional application method, without limit to spraying, dusting, broadcast spreading, or drop spreading. CPC may be applied in its dry form or slurried with liquid for wet applications, including spraying. Hydrolyzed CPC may be applied in liquid form by methods such as spraying or may be dried or dried onto/with another compound and applied in a dry form.

A deodorizing compound may be also be applied with CPC. In one aspect of the invention, the deodorizing compound is applied simultaneously with the CPC. One method of applying the deodorizing compound is to mix the compound in a dry form with the CPC prior to application. Optionally, the CPC and hydrolyzed CPC may optionally be slurried in a liquid in the presence of compounds prior to application. Examples of such compounds, without limit, include cyclodextrins and alcohols.

The color of CPC may be modified prior to or during application. For example, during application of CPC to turf grass, a blue or green dye may be applied to CPC to provide a color more similar the application and of better visual quality. The dye maybe applied to the CPC prior to application or mixed in a slurry of CPC during the application.

In another embodiment, CPC can be substituted for CGM as a nutrient in mushroom cultivation. CPC provides a nutrient source to the growth media as described herein U.S. Pat. No. 5,759,223. Briefly, a CPC described herein provides an all-natural, non-chemically modified supplement for increasing the growth and/or crop yield of fungi in a growth medium having a protein content of about 60% or greater of the total weight percent.

In accordance with the present invention, there may be employed conventional chemistries, biochemistries and microbiological techniques within the skill of the art. Such techniques are explained fully in the literature. The invention will be further described in the following examples, which do not limit the scope of the invention described in the claims.

### EXAMPLES

### Example 1

### Evaluation of CPC

Four samples of corn gluten were received for functional property analyses and were labeled A, B, C and D. Samples A and B were CPC and Samples C and D were corn gluten meal.

Proximate analysis was performed for moisture, fat, protein and ash. The results of this analysis are shown in Table 1 and are reported on a dry weight basis (with the exception of moisture). The pH of the samples was determined by slurrying the protein samples in an equal mass of distilled water, allowing to equilibrate for 10 minutes, then measuring pH with a pH probe-meter. Proximate analysis was performed using Official Methods of the AOAC International. Moisture was determined by AOAC 935.29; fat by AOAC 954.02; protein by AOAC 990.03; and ash by AOAC 942.05.

TABLE 1

| | A | B | C | D |
|---|---|---|---|---|
| Moisture | 5.37 | 6.16 | 5.91 | 4.69 |
| Protein | 78.14 | 81.05 | 68.32 | 67.31 |
| Fat | 8.95 | 5.54 | 8.06 | 6.24 |
| fat:protein ratio | 0.115 | 0.068 | 0.118 | 0.093 |
| Ash | 0.95 | 1.54 | 1.27 | 1.29 |
| ash:protein ratio | 0.012 | 0.019 | 0.019 | 0.019 |
| pH | 5.6 | 5.5 | 3.9 | 4.1 |

Solubility of CPC was tested in water and ethanol solutions. Solutions were prepared with enough CPC to produce a concentration equivalent of 5% protein suspended in different solvents to examine solubility. Water, water:denatured ethanol (in a 1:1 ratio), or denatured ethanol (97%) were used as the solvents. Protein analyses were done on the suspensions using the Bio-Rad Protein Assay. One ml aliquots of each suspension were centrifuged in an Eppendorf microcentrifuge for 10 minutes and the supernatants analyzed for dissolved protein using the Bio-Rad Protein Assay. Protein solubility indices were calculated as follows:

[protein content of supernatant/protein content of suspension]×100.

Table 2 shows that all four samples had relatively low solubility in water compared to, for example, globular proteins such as egg and purified soybean protein isolate.

Results indicated that the solubility of samples A and B were substantially increased in the water:ethanol solvent. The supernatant of sample D, however, tested at 2.9% and 2.1%, indicating a significant increase in solubility.

While there was some dissolved protein detected in ethanol-suspended samples A and B, the supernatants of samples C and D in the ethanol tested at 0.05% and 0.0%, respectively, indicating very low solubility for samples C and D.

The solubility in water was low for all four samples, although the presence of ethanol seemed to improve the solubility. In any event, a water:ethanol solvent appears to be a suitable solvent for samples A and B(CPC).

TABLE 2

| | A | B | C | D |
|---|---|---|---|---|
| Water | 2.03 | 1.96 | 5.04 | 3.01 |
| 1:1 Water:EtOH | 13.82 | 21.57 | 6.25 | — |
| EtOH | 16.35 | 9.39 | — | 0.0 |

The effect of pH on solubility of samples A and B was examined. The pHs of the aqueous suspensions prepared as described above for samples A and B were adjusted to each of the pHs shown below in Table 3 (i.e., 2, 4, 5.5, 7, 9 and 11) and an aliquot was drawn. The aliquot was centrifuged and analyzed for protein concentration, i.e., indicating relative solubility of the proteins at each pH. Results are shown in Table 3. There was a very small increase in solubility at alkaline pH for sample A, and little to no increase was detected for sample B.

TABLE 3

| pH | A | B |
|---|---|---|
| 2 | 0.250 | 0.075 |
| 4 | 0.150 | (below detection limits) |
| 5.5 | 0.112 | (below detection limits) |
| 7 | 1.193 | 0.062 |
| 9 | 0.612 | (below detection limits) |
| 11 | 1.325 | 0.168 |

More than one pH optimum for solubility was observed, but because these samples contain more than one type of protein, it is possible that the different proteins are solubilizing at different pH's.

### Example 2

### Evaluation of CPC

Three samples of CPC and three samples of corn gluten meal were sent to external laboratories for proximate analysis. Proximate analysis was performed using Official Methods of the AOAC International. Moisture was determined by AOAC 935.29; fat by AOAC 954.02 (acid hydrolysis) and AOAC 920.39 (ether extract); protein by AOAC 990.03; ash by AOAC 942.05, and total starch and sugars were determined by AOAC 996.11. Total starch was determined by official analytical method of the Corn Refiners Association, CRA G-28. Sugars are determined by the difference between total starch and sugars minus total starch. Sugars includes starch liquefaction products, higher sugars, and sugars, including those native to the wet milling streams captured in the CPC. Results of this analysis are shown in Table 4 and are reported on a dry weight basis (with the exception of moisture). The ratio of fat or ash to protein is calculated by the fat or ash content divided by the protein content on a dry compositional basis.

TABLE 4
Compositional Analysis of Corn Protein Concentrate
compared to Corn Gluten Meal

| | CPC 1 | CPC 2 | CPC 3 | CGM 1 | CGM 2 | CGM 3 |
|---|---|---|---|---|---|---|
| Moisture | 7.3 | 8.2 | 10.6 | 8.6 | 9.6 | 11.0 |
| Protein | 81.5 | 82.2 | 81.5 | 70.3 | 70.6 | 70.7 |
| Fat (Ether extract) | 2.42 | 2.56 | 2.44 | 1.25 | 1.11 | 1.39 |
| EE fat:protein ratio | 0.030 | 0.031 | 0.030 | 0.012 | 0.016 | 0.020 |
| Fat (acid hydrolysis) | 5.2 | 4.8 | 5.7 | 4.4 | 4.1 | 3.7 |
| AH Fat:protein ratio | 0.064 | 0.058 | 0.070 | 0.063 | 0.058 | 0.052 |
| Ash | 1.5 | 1.1 | 1.3 | 1.2 | 1.2 | 1.2 |
| ash:protein ratio | 0.018 | 0.013 | 0.016 | 0.017 | 0.017 | 0.017 |
| Total starch | 0.4 | 0.9 | 0.7 | 16.0 | 16.6 | 18.5 |
| Total starch & sugars | 5.4 | 5.7 | 6.6 | 18.5 | 19.2 | 20.5 |
| Sugars | 5.0 | 5.8 | 5.9 | 2.5 | 2.6 | 2.0 |
| Magnesium | 0.07 | 0.06 | 0.05 | 0.05 | 0.04 | 0.07 |
| pH | 5.6 | 5.5 | 5.5 | 4.4 | 4.6 | 4.3 |

CPC has a higher quantity of ether-extractable fats in comparison to the CGM. However, the total fat content of the samples as determined by acid hydrolysis is similar between CGM and CPC on a protein unit (ratio) basis. Although not bound by any particular mechanism, the process of making the CPC as described herein may release the fat so as to make it more available for extraction with ether. This higher level of “free” fats result in different functional and nutritional properties (e.g., extrusion processing functionality and digestibility) of CGC as compared to CGM. In addition, the greater accessibility of the fats and oils to solvents such as ether and hexane make the CPC material more easily defatted. The quantity of intact starch is decreased from 16.0-18.5% in CGM to 0.4-0.9% in CPC. The quantity of sugars and starch liquefact (higher sugars) is increased from 2.0-2.6% in CGM to about 5.0-5.9% in CPC (of the dry weight composition). The magnesium content of CPC is unexpectedly similar to the CGM and was not concentrated due to the removal of starch (e.g., the magnesium content is not significantly different on a mass basis and is lower on a protein basis than the CGM). As shown in Example 1, the pH of CGC is higher, at about 5.5-5.6, than CGM, at 3.9-4.6, and CGC has a more consistent pH for manufacturing benefits of pH control and cost of adjustment. When compared to CGM, CPC was found to contain fewer wet milling smells and odors; a panelist of judges and those familiar with the wet milling process found that CPC contained fewer smells commonly associated with the wet milling process in comparison to CGM and had a smell more similar to corn.

### Example 3

### Extraction of Fat from CPC

The concentration of protein in the CPC was increased through removal of the fats (i.e., defatting). One method of defatting is performed by passing hexane through a bed of CPC in an industrial solvent extractor. The hexane is applied in a countercurrent flow pattern to the movement of the newest to most fat extracted CPC. The CPC is desolventized after centrifuging or filtering using a desolventizing-toaster apparatus commonly found in oilseed and germ extraction plants. Examples of other solvents that can be used include hexane, isohexane, alcohols, and mixtures thereof. Alternatively, the solvent can be applied to the CPC and separated in a reflux or membrane separation devices. The solvent can be recovered through distillation to separate the oil from the solvent and the reclaimed solvent can be reused in the extraction process.

### Example 4

### Further Processing

The CPC is processed by heated refluxing in 2 N HCl for 1 hr at 90° C. to hydrolyze the protein. The hydrolyzed protein can be used as is as a higher solubility protein source applied in liquid form or can be dried before use.

### Example 5

### Water Absorption and Solubility Indeces

Water absorption index and water solubility index of CGM (1 and 2 samples obtained Dec. 4 and July 05) and CPC (1 and 2, prepared Feb. 4 and June 05, respectively), from a corn wet milling plant in the USA, was determined as outlined in American Association of Cereal Chemists (AACC) Official Methods 56-20 and also by Anderson et al., 1969, Cereal Science Today, 14(1):4-11. Additionally, the % solubilized protein was calculated as the calculated percent of the original samples protein content that was measured in the supernatant after centrifuging as measured by AACC using an Elementar Variomax nitrogen analyzer and a protein conversion factor of 6.25. The CPC product had a higher WSI than the CGM. WAI and the % protein in the supernatant were not statistically different.

TABLE 5
WAI and WSI in water by Protein Products

| Product | WAI (%) | WSI (%) | % Solubilized Protein |
|---|---|---|---|
| CGM 1 | 252 | 3 | 4.1 |
| CGM 2 | 260 | 3 | 5.0 |
| CPC 1 | 253 | 6 | 5.7 |
| CPC 2 | 307 | 5 | 2.6 |

### Example 6

### Protease Digestibility

About 3 grams of protein material was suspended in 30 grams of water, to which an amount of enzyme was added and the mixture was incubated in a shaking waterbath at 50° C. for 20 hrs. Treatment 1 was an amount of 3 microliters of GC106 protease enzyme (Genencor International, Palo Alto, Calif., USA) was added to each mixture of CPC or CGM and water (pH adjusted to 4.3). Treatment 2 was an amount of 50 microliters of each of GC106 and Proteinase T was added to each mixture (pH adjusted to 4.3). After incubating 20 hrs, each mixture was centrifuged at 4000 g for 15 minutes and the supernatant was tested for soluble protein content by AOAC 990.03. No significant difference in protein digestibility was observed between CGM and CPC. Total protein digested by the proteases appeared dependant on both concentration and the type of protease used.

TABLE 6
% of Original CGM or CPC Protein (Nitrogen × 6.25)
Released into Solution with Protease Treatment

| | Enzyme Treatment (20 hr, 50° C., pH 4.3) | |
|---|---|---|
| Product | Treatment 1: GC106 | Treatment 2: Proteinase T + GC106 |
| CGM 1 | 11 | 27 |
| CGM 2 | 14 | 28 |
| CPC 1 | 11 | 28 |
| CPC 2 | 8 | 25 |

### Example 7

### Further Processing into a Protease Hydrolyzate

The CPC is processed by treatment with proteases. A slurry of fully and partially hydrolyzed protein is created due to the mixture of protein types present in corn gluten. The glutelin and water-soluble proteins are hydrolyzed more quickly and to a greater extent than the zein proteins. The liquids may optionally be dried as a slurry mass may optionally be centrifuged or filtered to separate the different components. The hydrolyzed protein may be applied as a herbicide, fertilizer, or nutrient media in liquid form or in dried form, alone or as a component ingredient.

### Example 8

### DeOdorizing

A mixture of cyclodextrins (alpha-, beta-, and propylated-beta-) are applied to the filtered cake material of CPC. The cyclodextins may be applied in dry form and mixed with the cake or sprayed onto the cake in wet form. The treated cake is then dried in the presence of the cyclodextrin. The dried CPC has substantially less corn and/or wet milling associated odors than the untreated material.

### Example 9

### DeOdorizing

An aqueous and alcohol solution of cyclodextrins was applied to finished CPC by a spray application at two concentrations of about 0.1% and about 2% on a wt/wt basis. About 25 grams of CPC was placed in 150 ml sealed containers. Solutions of cyclodextrin or a control of water was applied to the CPC. The CPC was mixed and allowed to equilibrate for 5 minutes in a sealed container. The ability of the treatment to substantially reduce the amount of odor in the head-space within the container was tested by a difference testing based on sniff testing using human judges. A panel of 6 judges unanimously found that the CPC treated with cyclodextrins had a significantly and substantially reduced odor commonly associated with the wet milling process as compared to original samples or those treated with an equivalent amount of water. The treated product was described as having a corn-like note when applied at very low levels of about 0.1% to having a bland, lack of smell when the solution was applied at about 2%. The treated CPC may be dried. Alternatively, similar results are expected when applying cyclodextrins to CPC in dry form.

### Example 10

### Herbicide and Fertilizer Application

CPC was applied to a narrow blade fescue turfgrass lawn at the rate of 8 lbs/1000 sq. ft. in three applications over the course of three months (June, July, and August). No other commercial fertilizer or herbicide was applied during the summer growing season. No weed growth was observed during and after the application period prior to fall freezing conditions. Areas of the lawn that CPC was applied to showed a deeper green turfgrass growth and a higher visual quality to the lawn. CPC was determined to have herbicidal and fertilizer properties and improved the quality of the lawn to the satisfaction of the owner and user.

### Example 11

### Microbial Stability

Four samples of CPC and a sample of corn gluten meal, obtained from a plant location in the United States, were sent to external laboratories for microbiological and water activity analysis. CPC had a less bacterial counts as determined by a Standard Plate Count test conforming to AOAC Official Methods of Analysis, sec. 966.23 as shown in Table 7. Standard plate counts of other CGM and CPC samples are expected to show similar differences.

TABLE 7
Comparison of Standard Plate Counts of CPC compared to CGM

| Sample | Standard Plate Count |
|---|---|
| Corn Gluten Meal | 2000 |
| CPC Lot #1 | 450 |
| CPC Lot #2 | 220 |
| CPC Lot #3 | 460 |
| CPC Lot #4 | 330 |

### Example 12

### Caustic Solubility Index

CPC and CGM samples from Example 5 were tested for solubility in 0.5 N sodium hydroxide based on methods and materials used in Example 5. CPC and CGM were placed in 0.5 N sodium hydroxide (NaOH) and mixed for 1 hr. The samples were centrifuged at 4000×g for 10 min and the supernatant collected. The % solubilized protein was measured in the supernatant after centrifugation as measured by AOAC 990.03 and was expressed as the percent of the protein content in the original samples. CPC unexpectedly had lower protein solubility (protein released into the solution) in a solution of 0.5 N sodium hydroxide (NaOH) than did the CGM. Results are shown in Table 8.

TABLE 8
Solubilized Protein in 0.5 N NaOH of CGM and CPC

| Product | % Solubilized Protein |
|---|---|
| CGM 1 | 74.3 |
| CGM 2 | 74.3 |
| CPC 1 | 28.8 |
| CPC 2 | 27.5 |

### Example 13

### Sodium Dodecyl Sulfate Solubility Index

CPC and CGM solubility in a solution of 1% SDS was tested based on methods and materials used in Example 5. CPC and CGM were placed in 1% SDS and mixed for 1 hr. The samples were centrifuged at 4000×g for 10 min and the supernatant collected. The % solubilized protein was measured in the supernatant after centrifugation as measured by AOAC 990.03 and was expressed as the percent of protein content in the original sample. Slightly less protein (e.g., nitrogen) was released into solution from the CPC as compared to the CGM.

TABLE 9

| Product | % Solubilized Protein |
|---|---|
| CGM 1 | 11.8 |
| CGM 2 | 13.9 |
| CPC 1 | 10.4 |
| CPC 2 | 8.4 |

### Example 13

### Oil Solubility and Adsorption Index

About 7 grams of protein material was suspended in 20 grams of corn oil and tumbled in 50 ml centrifuge tubes for 1 hour. The tubes were then centrifuged at 4500 g for 15 minutes and the supernatant oil was poured off. An amount of 20 grams of water was added and the pellet was resuspended by vortexing for 15 sec. The mixture was again centrifuged at the above conditions. The oil suspended was removed, dried and weighed and Oil Binding Index=weight of oil recovered/weight of protein material(db)*100. The supernatant water was further poured off the pellet and the pellet was dried. The increase in dry basis weight between the initial material and the dried pellet was determined and the Oil Adsorption Index=Weight increase of pellet(db)/weight of protein material(db)*100. Increase of dry basis weight of the protein material was assumed to be oil adsorbed during treatment. CPC adsorbed less oil onto particle surfaces than CGM control. Results are shown in Table 29.

TABLE 10
% Corn Oil Adsorbed and Bound by Protein Products

| Product | OAI | Oil Binding Index |
|---|---|---|
| CGM 1 | 109 | 1151 |
| CGM 2 | 111 | 1300 |
| CPC 1 | 103 | 1335 |
| CPC 2 | 103 | 1168 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A herbicide product comprising a corn protein concentrate, wherein the corn protein concentrate is prepared by a process comprising:
contacting one or more protein containing materials with one or more wet-mill streams and one or more carbohydrases to produce at least one protein concentrate and at least one aqueous stream containing water-soluble carbohydrates; and
separating the protein concentrate from the aqueous stream containing water-soluble carbohydrates.

2. The herbicide product of claim 1, further comprising a base herbicide wherein the corn protein concentrate is incorporated on or in the base herbicide.

3. The herbicide product of claim 1, wherein the corn protein concentrate has higher pH than corn gluten meal.

4. The herbicide product of claim 1, wherein the corn protein concentrate has a pH of from about 4.8 to about 5.6.

5. The herbicide product of claim 1, wherein the corn protein concentrate does not have an acidification effect on soil pH.

6. The herbicide product of claim 1, wherein the corn protein concentrate provides a source of nitrogen nutrients to soil and plants.

7. The herbicide product of claim 1, wherein the process further comprises decoloring, bleaching, and/or reducing color-bodies present in the protein-containing material.

8. The herbicide product of claim 1, wherein the process further comprises contacting the corn protein concentrate with a deodorizing compound.

9. The deodorizing compound of claim 8, wherein the deodorizing compound is a cyclodextrin.

10. The cyclodextrin of claim 9, wherein the cyclodextrin is selected from the group of alpha-cyclodextrin, beta-cyclodextrin, gamma-cyclodextrin, hydroxylpropyl-beata-cyclodextrins, and/or mixtures thereof.

11. A herbicide product of claim 1, wherein herbicide has less odor than a herbicide made with corn gluten meal.

12. The herbicide product of claim 1, wherein the herbicide has a lower application rate than a herbicide made with corn gluten meal.

13. The herbicide product of claim 12, wherein the application rate from about 5 to about 50 lbs/1000 sq ft.

14. The herbicide product of claim 1, wherein the herbicide has a lower application rate than a herbicide made with corn gluten meal and has from about 15 to about 25% less mass volume.

15. A fertilizer product comprising a corn protein concentrate, wherein the corn protein concentrate is prepared by a process comprising:
contacting one or more protein containing materials with one or more wet-mill streams and one or more carbohydrases to produce at least one protein concentrate and at least one aqueous stream containing water-soluble carbohydrates; and
separating the protein concentrate from the aqueous stream containing water-soluble carbohydrates.

16. The fertilizer product of claim 15, further comprising a base fertilizer wherein the corn protein concentrate is incorporated on or in the base fertilizer.

17. The fertilizer product of claim 15, wherein the corn protein concentrate has higher pH than corn gluten meal.

18. The fertilizer product of claim 15, wherein the corn protein concentrate has a pH of from about 4.8 to about 5.6.

19. The fertilizer product of claim 15, wherein the corn protein concentrate does not have an acidification effect on soil pH.

20. The fertilizer product of claim 15, wherein the corn protein concentrate provides a source of nitrogen nutrients to soil and plants.

21. The fertilizer product of claim 15, wherein the process further comprises decoloring, bleaching, and/or reducing color-bodies present in the protein-containing material.

22. The fertilizer product of claim 15, wherein the process further comprises contacting the corn protein concentrate with a deodorizing compound.

23. The deodorizing compound of claim 22, wherein the deodorizing compound is cyclodextrin.

24. The cyclodextrin of claim 23, wherein the cyclodextrin is selected from the group of alpha-cyclodextrin, beta-cyclodextrin, gamma-cyclodextrin, hydroxylpropyl-beata-cyclodextrins, and/or mixtures thereof.

25. The fertilizer product of claim 15, wherein the fertilizer product has less odor than a fertilizer made with corn gluten meal.

26. The fertilizer product of claim 15, wherein the herbicide has a lower application rate than a fertilizer made with corn gluten meal.

27. The fertilizer product of claim 26, wherein the application rate from about 5 to about 50 lbs/1000 sq. ft.

28. The fertilizer product of claim 1, wherein the herbicide has a lower application rate than a fertilizer made with corn gluten meal and has from about 15 to about 25% less mass volume.

29. A growth media product comprising a corn protein concentrate, wherein the corn protein concentrate is prepared by a process comprising:
contacting one or more protein containing materials with one or more wet-mill streams and one or more carbohydrases to product at least one protein concentrate and at least one aqueous stream containing water-soluble carbohydrates; and
separating the protein concentrate from the aqueous stream containing water-soluble carbohydrates.

30. The growth media product of claim 29, further comprising a base media wherein the corn protein concentrate is incorporated on or in the base media.

31. The growth media product of claim 29, wherein the growth media product is a used in the cultivation of mushrooms.
